(19) United States
(12) Patent Application Publication
(10) Pub. No.: US 2008/0161851 A1
(43) Pub. Date: Jul. 3, 2008

(54) Resorbable Suture Material

(76) Inventors: Erich Odermatt, Schaffhausen (CH); Jurgen Egloff, Tuttlingen (DE); Rainer Bargon, Mengen (DE); Erhard Muller, Stuttgart (DE)

Correspondence Address: NATH & ASSOCIATES, 112 South West Street, Alexandria, VA, 22314, US

(21) Appl. No.: 11/883,531

(22) PCT Filed: Feb. 3, 2006
(86) PCT No.: PCT/EP06/00935

§ 371 Date: Feb. 21, 2008

(30) Foreign Application Priority Data
Feb. 4, 2005 (DE) 10 2005 006 718.2

(51) Int. Cl. A61B 17/04

(52) U.S. Cl. 606/230

(57) ABSTRACT

The invention concerns absorbable, thermally steam-treated, surgical suture based on polyglycolic acid and/or lactide copolymer comprising rapid degradation and rapid decrease in knot-pull breaking strength under in vivo conditions and an initial knot-pull breaking strength which is high for the rapid degradation.

This invention relates to absorbable, surgical suture based on polyglycolic acid or on a lactide copolymer. The suture exhibits accelerated degradation in vivo. The present invention further relates to a process for producing acceleratedly absorbable, surgical suture.

Absorbable sutures are capable of being absorbed over time after implantation in the body. “Conventional” absorbable sutures now include in particular polymers and copolymers of α-hydroxy acids, in particular of glycolic acid and lactic acid.

Polylactides, as will be known, are biodegradable polyesters based on lactic acid. They usually exist in two optical isomers, poly-L-lactic acid and poly-D,L-lactic acid. While the former is partly crystalline, relatively hard and brittle, the latter tends to be amorphous and flexible. In general, the degradation behavior of a polylactide can be pre-set by defined copolymerization of the two isomers.

In the body, absorbable suture based on the specified polymers and copolymers is usually degraded by hydrolysis, although polylactides also degrade enzymatically. The resulting degradation products, glycolic acid and lactic acid, are metabolized in the body or, as the case may be, excreted in the unmetabolized state.

An example of a tried and tested absorbable suture is Safil®, a suture marketed by the applicant. Safil® is a mid-term absorbable, synthetic, braided and coated suture composed of polyglycolic acid (polyglycolide), available for example with a violet color and also without colorant. Safil® is predictably and reliably degraded by hydrolysis and absorbed.

An example of suture with the building blocks lactic acid and glycolic acid is the copolymer of glycolide and lactide in a ratio of 9:1 that is marketed by Ethicon under the designation Vicryl®.

Tyco markets absorbable, braided suture under the trade name Polysorb®; this again comprises a copolymer based on glycolide and lactide.

Absorbable sutures based on lactide copolymers among others are described in EP 999227 A2.

Medical indications where a surgeon's stitch will have done its job after a short period frequently utilize sutures which are more rapidly absorbed in the body than is the case with conventional absorbable sutures.

Various ways are known to shorten the absorption time of conventional absorbable suture. The most important will be briefly outlined in what follows.

In a widely used process, the suture is irradiated, preferably with gamma radiation, during or after its fabrication. The radiation creates defects in the polymeric structure of the suture and thereby leads to a faster loss of breaking strength and a shorter absorption time for the suture in vivo. Such a treatment is associated with high capital cost and inconvenient workplace safety precautions. Nor is the initial knot-pull breaking strength (depending on thread gage) satisfactory in an irradiation process. Moreover, irradiation of colored suture may present problems in that, for example, the violet dye D&C violet No. 2 is not persistent under irradiation with gamma rays.

DE 197 02 708 describes a process for hydrolyzing suture in various buffering systems. The process is preferably carried out in the course of the manufacturing operation following spinning, braiding, drawing and an annealing step which is carried out at temperatures between 70° and 120° C. In this annealing step, absorbable surgical suture is incubated in an incubating bath at a temperature in the range from 30° C. to 65° C. for a period of at least 10 hours, preferably more than 30 hours. The choice of the process parameters of temperature, time and pH influences the degradability of the resulting suture and also loss of breaking strength in vitro and in vivo. The parameters mentioned therefore have to be monitored very closely In addition, the process, taking up to several days, is very time intensive.

WO 2004/050127 likewise describes a process wherein bioabsorbable material like the abovementioned Polysorb® is subjected to partial degradation by hydrolysis. To this end, the material is introduced into an environmental chamber in which it rests for a period of preferably 5 to 8 days at 20% to 70% relative humidity (preferably between 45% and 55%) and a maximum temperature of 93° C. (200° F.). The temperature range between 52° C. and 57° C. is specified as preferred. The process is proposed for the treatment of surgical devices in general and suture in particular. Again, the particular disadvantage is the long time needed to carry out the process.

It is an object of the present invention to provide an absorbable suture whose knot-pull breaking strength in vivo, i.e., post implantation, decreases rapidly. The present invention also has for its particular object to provide a process for producing acceleratedly absorbable, surgical suture that avoids the identified disadvantages of existing processes. The process shall in particular be simpler, faster and hence cheaper to carry out than existing processes.

This object is achieved according to the present invention by absorbable, thermally steam-treated, surgical suture based on polyglycolic acid and/or lactide copolymer comprising rapid degradation and rapid decrease in knot-pull breaking strength under in vivo conditions and an initial knot-pull breaking strength which is high for the rapid degradation.

The inventors found that sutures composed of polymers based on glycolic acid and/or polymers based on lactic acid exhibit the desired properties after a steam treatment, whereas sutures composed of polymers and copolymers of other hydroxy acids fail in this regard.

Suitable lactide copolymers are described in particular in EP 999227 A2 and have a majority fraction of lactide. The known polyglycolic acid comprises a polyester based on glycolic acid. Present invention suture based on polyglycolic acid is particularly preferred. The present invention's suture based on polyglycolic acid may further comprise a suture which is preferably wholly crystalline.

It is preferred that the knot-pull breaking strength of present invention suture based on polyglycolic acid decreases within 5 days to less than 65% of the initial knot-pull breaking strength.

It is further preferred that the knot-pull breaking strength of present invention suture based on polyglycolic acid decreases within 14 days to less than 10% of the initial knot-pull breaking strength.

Present invention suture based on lactide copolymer is degraded retardedly compared with present invention suture based on polyglycolic acid. It is preferred that the knot-pull breaking strength decreases within 14 days to 75% to 90% of the initial knot-pull breaking strength.

It is further preferred that the knot-pull breaking strength of present invention suture based on lactide copolymer decreases within 28 days to 60% to 75% of the initial knot-pull breaking strength.

The aforementioned initial knot-pull breaking strength of present invention suture is preferably at least 65% of the knot-pull breaking strength of non-steam-treated suture. This holds for suture based on polyglycolic acid and also for suture constructed on the basis of a lactide copolymer.

Present invention suture is preferably obtainable by a thermal treatment of hydrolyzable suture with water vapor. The thermal treatment is preferably effected under superatmospheric pressure. It is believed that the thermal treatment with water vapor leads to a partial degradation of the polymer chains of the present invention's suture by hydrolysis. This belief is endorsed by a higher level of acid groups in steam-treated suture compared with non-steam-treated material. Samples of steam-treated Safil® were found to have a free glycolic acid content of preferably 20 to 45 mg/g, in particular 30 to 35 mg/g, irrespective of thread gage.

Knot-pull breaking strength has particular significance, especially practical significance, as a characteristic of a thread. Suture threads are usually implanted in the knotted state. A knot will always constitute the weakest point of a stitch because of the shearing forces which arise. By definition, the knot-pull breaking strength is the force at which the thread in the knot (which consists of a tightly pulled loop) breaks. The straight-pull breaking strength of a suture is by contrast the force at which an unknotted thread breaks when subjected to a tensile force in the direction of its longitudinal axis. The initial knot-pull breaking strength of the present invention's suture is astonishingly high given the accelerated degradability.

The decrease in the aforementioned knot-pull breaking strength which a thread undergoes in the course of its degradation in vivo must not be equated with the degradation of the surgical suture. The thread will only perform its function as long as it has its breaking strength. A thread is in the fully degraded state when its material is no longer detectable in the tissue. However, it will have lost its breaking strength long before that point in time.

Percentage and absolute data reported herein concerning the knot-pull breaking strength of present invention suture at various stages are values determined in a simulated in vivo degradation. To this end, the suture was placed at room temperature in a Sörensen type buffering solution, removed at certain time intervals (for example, after 5, 7, 10 and 14 days) and examined. Placing suture in a Sörensen type buffering solution is a commonly employed method of simulating degradation in vivo. In the present case, an aqueous KH₂PO₄ and Na₂HPO₄ solution which has a pH of 7.4 at 25° C. was used. All data reported herein concerning the knot-pull breaking strength of present invention suture following a degradation in vivo were determined in this way. A further commonly employed method of simulating degradation in vivo consists in storing the suture in a sodium chloride solution.

The initial knot-pull breaking strength of present invention suture does of course also depend in particular on the thread gage of the suture, which will hereinbelow be categorized in accordance with the classification promulgated by the United States Pharmacopeia (USP). Table 1 shows preferred ranges for the initial knot-pull breaking strength of present invention suture based on polyglycolic acid for different thread gages, each compared with the knot-pull breaking strength of the respective thread before the treatment by the present invention's process (all data in newtons).

TABLE 1
Initial knot-pull breaking strength of inventive
suture based on polyglycolic acid for different
thread gages, each compared with knot-pull breaking
strength of respective thread before treatment by inventive process

| | Knot-pull breaking | Initial knot-pull |
|---|---|---|
| Thread | strength before steam | breaking strength [N] |
| gage | treatment [N] | (after steam treatment) |
| USP 2 | 90-110, especially 95-105 | 65-95, especially 70-95 |
| USP 1 | 60-90, especially 70-80 | 50-70, especially 55-70 |
| USP 0 | 40-65, especially 45-60 | 35-50, especially 40-50 |
| USP 2-0 | 35-55, especially 40-50 | 20-40, especially 25-40 |
| USP 3-0 | 15-35, especially 22-32 | 15-27, especially 18-27 |
| USP 4-0 | 10-25, especially 15-22 | 10-20, especially 14-20 |
| USP 5-0 | 9-15, especially 10-13 | 5-11, especially 7-10 |

In a preferred embodiment, present invention suture based on polyglycolic acid and having a thread gage of USP 2 has an initial knot-pull breaking strength of 65 to 95 N, especially 70 to 95 N, after steam treatment.

In a further preferred embodiment, present invention suture based on polyglycolic acid and having a thread gage of USP 1 has an initial knot-pull breaking strength of 50 to 70 N, especially 55 to 70 N, after steam treatment.

In a further especially preferred embodiment, present invention suture based on polyglycolic acid and having a thread gage of USP 0 has an initial knot-pull breaking strength of 35 to 50 N, especially 40 to 50 N, after steam treatment.

In a further especially preferred embodiment, present invention suture based on polyglycolic acid and having a thread gage of USP 2-0 has an initial knot-pull breaking strength of 20 to 40 N, especially 25 to 40 N, after steam treatment.

In a further especially preferred embodiment, present invention suture based on polyglycolic acid and having a thread gage of USP 3-0 has an initial knot-pull breaking strength of 15 to 27 N, especially 18 to 27 N, after steam treatment.

In a further especially preferred embodiment, present invention suture based on polyglycolic acid and having a thread gage of USP 4-0 has an initial knot-pull breaking strength of 10 to 20 N, especially 14 to 20 N, after steam treatment.

In a further especially preferred embodiment, present invention suture based on polyglycolic acid and having a thread gage of USP 5-0 has an initial knot-pull breaking strength of 5 to 11 N, especially 7 to 10 N, after steam treatment.

Considering all thread gages together, the value of the initial knot-pull breaking strength is preferably in the range between 7 N and 95 N.

Suture consisting essentially of polyglycolic acid is particularly preferred according to the present invention. In a further development, present invention suture consisting of. polyglycolic acid is particularly preferred when it has a viscosity number in the range between 0.45 dl/g and 0.65 dl/g. For comparison, untreated suture consisting of polyglycolic acid generally has a viscosity number in the range between 0.9 dl/g and 1.0 dl/g.

Present invention suture based on polyglycolic acid may have a different mass average molecular weight (in daltons) compared with untreated suture. Preferably, the present invention's suture based on polyglycolic acid has a mass average molecular weight (in daltons) which is lower, in particular 20% to 30% lower, than untreated suture.

The suture according to the invention may comprise monofil, multifil, especially braided monofil, or else pseudomonofil, surgical suture. It preferably comprises multifil suture. In principle, it may also comprise multifil suture fabricated from threads of different materials, for example from threads based on polyglycolic acid and from threads based on a lactide copolymer.

It is particularly preferred for the present invention's suture to have a coating. An example of a preferred coating is a coating of a copolymer of glycolide, ε-caprolactone and trimethylene carbonate. Coatings containing a salt of a fatty acid, for example magnesium stearate or calcium stearate, are also conceivable.

Present invention suture is not impaired in its handling or in its appearance compared with untreated suture. The process is also applicable with particular advantage to dye-incorporating suture (for example the aforementioned violet Safil®). Whereas the color, as mentioned above, is affected in particular by conventional treatment with gamma radiation, this problem only occurs to a distinctly reduced extent and typically not at all under treatment in the course of the present invention's process.

The present invention further provides a process for producing rapidly absorbable, surgical suture, in particular for producing above-described, present invention suture. In the process, hydrolyzable suture, preferably composed of a polymer based on an α-hydroxy acid, especially of polyglycolic acid and/or lactide copolymer, is subjected to a thermal treatment with water vapor.

It is preferable according to the present invention that the treatment is carried out with water vapor which is free of admixtures which may actively or passively contribute to the abovementioned, partial degradation of the polymer chains of the suture to be treated. However, it is also possible for the water vapor to be admixed for example with additives having an exclusively therapeutic effect. It is particularly preferred, however, for the water vapor to be used free of any treating agents or other admixtures in pure, saturated form.

Preferably, the thermal treatment in the process of the present invention is carried out at a treatment temperature in the range between 100° C. and 150° C., preferably in the range between 105° C. and 150° C. and especially in the range between 120° C. and 150° C. The temperature range between 120° C. and 140° C. is particularly preferred.

It is further preferred for the thermal treatment to be carried out at elevated pressure, preferably in the range between 1 bar and 5 bar and especially between 2 bar and 5 bar. The range between 2 bar and 3 bar is particularly preferred.

In a preferred embodiment of the process according to the present invention, the thermal treatment is carried out for a duration of 4 minutes up to 30 minutes. In a further development, it is preferred for it to be carried out for a period of 4 min up to 22 min. Especially the short treatment duration constitutes an immense advantage compared with conventional processes, especially the abovementioned processes in buffering systems or in an environmental chamber.

It is preferred for the treated suture to be dried after the thermal treatment with water vapor. The drying of the suture is effected in particular at elevated temperature, more preferably in the range between 60° C. and 140° C. In a particularly preferred embodiment of the process according to the present invention, the drying is effected under reduced pressure, preferably in the range between 0.05 bar and 0.2 bar and especially in the range between 0.05 bar and 0.1 bar. Depending on the drying conditions, in particular the drying time, the values measured for knot-pull breaking strength, especially within 14 days, can be subject to certain fluctuations. However, the characteristic course of the decrease in knot-pull breaking strength, especially within 14 days, as described in the preceding embodiments of the present invention, remains essentially unaffected.

Since the knot-pull breaking strength is a thread characteristic which is particularly dependent on the gage (thickness or diameter) of the thread, it can be sensible according to the present invention to use the specific knot-pull breaking strength (diameter-corrected knot-pull breaking strength, units: N/mm²) to characterize the present invention's suture. Characterizing the present invention's suture in terms of its specific knot-pull breaking strength reveals that the suture has a high specific initial knot-pull breaking strength (diameter-corrected initial knot-pull breaking strength, units: N/mm²) for its degradation and the specific knot-pull breaking strength preferably decreases rapidly, especially within 14 days, the decrease in knot-pull breaking strength being at least partly linear. Characterizing in terms of specific knot-pull breaking strength permits in particular a comparison with other sutures, especially in various thread gages.

It is particularly advantageous for the present invention's suture, especially suture based on polyglycolic acid, to have a lower specific knot-pull breaking strength compared with a corresponding suture treated by conventional sterilization processes, especially irradiation. Preferably, the present invention's suture has a lower specific knot-pull breaking strength than suture correspondingly treated with gamma (γ) irradiation using customary doses of irradiation, especially in a range between 30 and 70 kGy (kilograys), preferably a dose of about 32 kGy or about 64 kGy. True, the specific knot-pull breaking strength of sutures can in principle be reduced by higher doses of radiation, but their packaging imposes limits to the irradiation of suture. Conventional packaging materials can be exposed to a maximum dose of about 70 kGy.

It can further be sensible according to the present invention to characterize the suture in terms of its specific initial knot-pull breaking strength. The present invention's suture based on polyglycolic acid, especially Safil®, preferably has a higher specific initial knot-pull breaking strength compared with suture based on polyglycolic acid and lactic acid, for example Vicryl® and Vicryl® Rapid.

The thermal treatment with water vapor is preferably carried out in an autoclave. Preferably, the threads are not only steam-treated but also subsequently dried in the autoclave. The temperature in the autoclave is preferably controllable, like the pressure. The autoclave is preferably of adequate size to be able to accommodate a major amount of suture. It is preferred that the water vapor needed to carry out the process of the present invention can be supplied directly. But it is also conceivable, as an alternative, to generate the water vapor by vaporization in the autoclave.

In a particularly preferred embodiment of the process according to the present invention, the thermal treatment is carried out with water vapor essentially free of air. More particularly, it is effected in a purely water vapor atmosphere. This can be realized by evacuating the autoclave before the steam treatment and also, if appropriate, flushing it with an inert gas or some other suitable fluid.

It is a particular advantage for the initial knot-pull breaking strength of the absorbable surgical suture produced to be settable as a function of thread gage by the process of the present invention. This setting is preferably effected through suitable choice of the treatment duration, whereby the fraction of partially degraded polymer chains in the treated suture is influencable. In principle, however, the initial knot-pull breaking strength of the absorbable surgical suture produced can also be influenced through variation of the parameters of pressure and temperature or else by simultaneous variation of at least two of the parameters mentioned.

The treatment of hydrolyzable suture by the process of the present invention provides suture whose knot-pull breaking strength is somewhat lower than that of the starting material. Typically, knot-pull breaking strength decreases by about 10% as a result of the treatment. Deviations from this value are possible depending on material, constitution, diameter of suture prior to treatment and, as the case may be, further parameters.

It was further determined that a longitudinal shrinkage of the treated suture occurs in the process of the present invention unless it is prevented, for example by clamping the threads. This longitudinal shrinkage, if allowed, occurs in combination with an increase in the diameter of the treated suture. The longitudinal shrinkage, unless prevented, is also dependent on the choice of starting materials, including in particular their constitution and diameter. The resulting suture can be up to 40% shorter than the starting material. Typically, shrinkage is not les than 2.5%.

Preferably, the process of the present invention is used to treat suture consisting essentially of polyglycolic acid. In the case of suture consisting essentially of polyglycolic acid, a diameter increase by about 7% was observed for a 5 min steam treatment at 121° C. on Safil® violet USP 5-0, for example.

In another embodiment, the process of the present invention is used to treat suture consisting essentially of lactide copolymer. The diameter increase mentioned can in this case even be up to 63% in some instances, for example for a lactide copolymer as described in EP 999227 A2, which has a 75% hard fraction and a 25% soft fraction. In the specific case of this block copolymer consisting of a soft-hard combination, the hard segment was composed of 95% by weight L-lactide and 5% by weight trimethylene carbonate (TMC), while the soft segment was composed of 45% by weight ε-caprolactone, 45% by weight trimethylene carbonate and 10% by weight L-lactide.

The process of the present invention can be used to treat multifil, monofil and also pseudomonofil suture. In principle, the constitution of the suture in this regard is essentially uncritical.

The process of the present invention can also treat suture having a coating, in particular a coating of a copolymer of glycolide, ε-caprolactone and trimethylene carbonate. It is in principle conceivable to influence the process through appropriate choice of the coating and to influence the degradation behavior of suture treated by the process of the present invention.

Colored suture is likewise treatable by the process of the present invention, as already mentioned elsewhere.

In a further preferred embodiment, the process of the present invention comprises treating ready-to-use suture, including in particular suture provided with a needle. In contradistinction to some other processes, the process of the present invention has universal applicability in that it cannot only be integrated into the manufacturing operation but also, as emphasized here, be applied to ready-to-use suture.

Any sutures obtained or obtainable by a process according to the present invention likewise form part of the subject matter of this invention.

A thread treated by the process of the present invention can in principle be stored for a very long time.

Further details and features of the invention will become apparent from the subsequent description of preferred embodiments in combination with the drawings, the examples and the subclaims. Individual features of the invention may be actualized in these embodiments alone or in combination with other features. The preferred embodiments described are to be merely understood as descriptive disclosure, not in any way limiting. All the drawings are hereby made content of this description by express reference.

### THE DRAWINGS SHOW

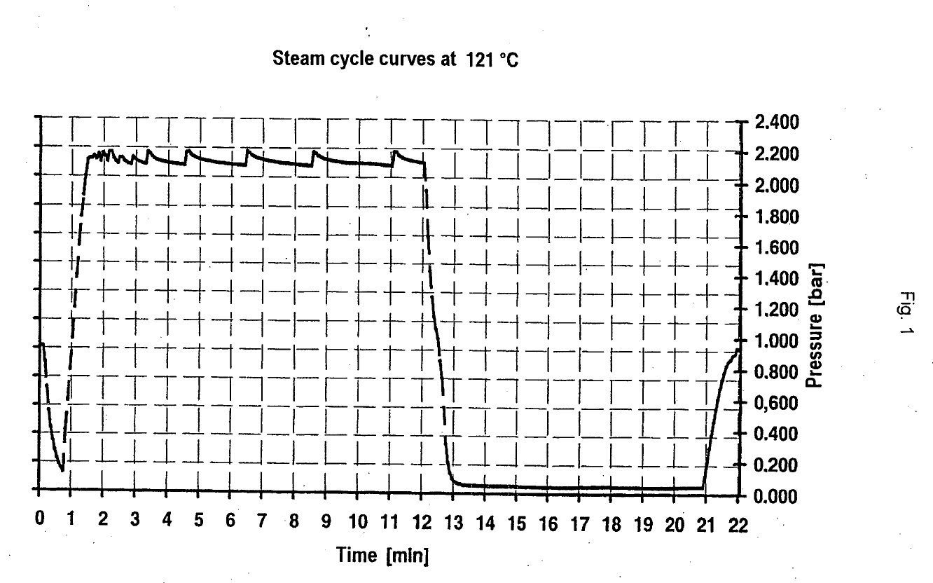

In FIG. 1 a steam cycle typical for an embodiment of the inventive process.

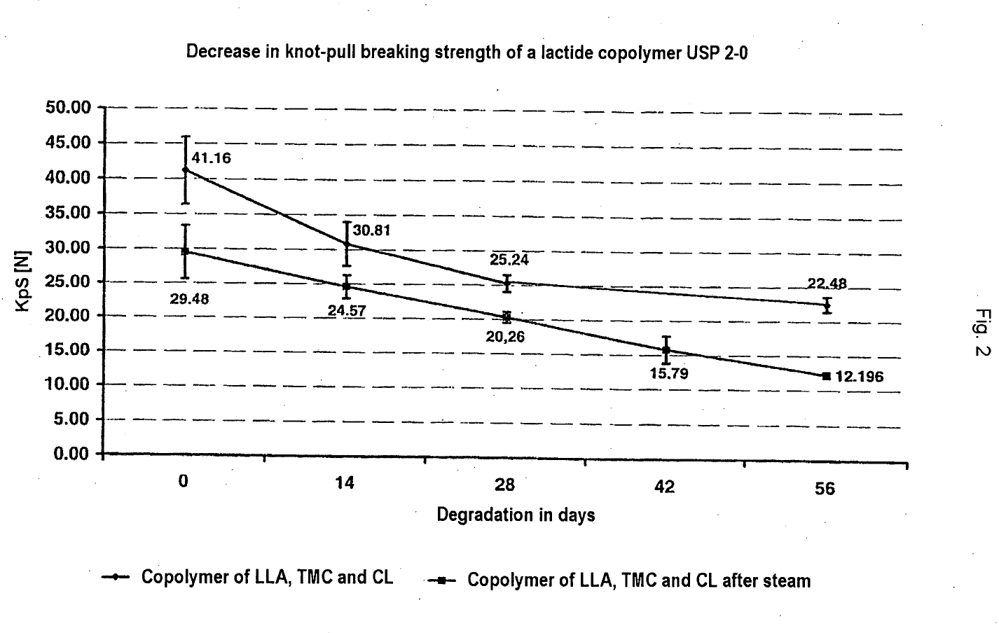

In FIG. 2 the decrease in the knot-pull breaking strength of a monofil suture of lactide copolymer after steam treatment at 121° C. for a period of 5 min compared with untreated suture.

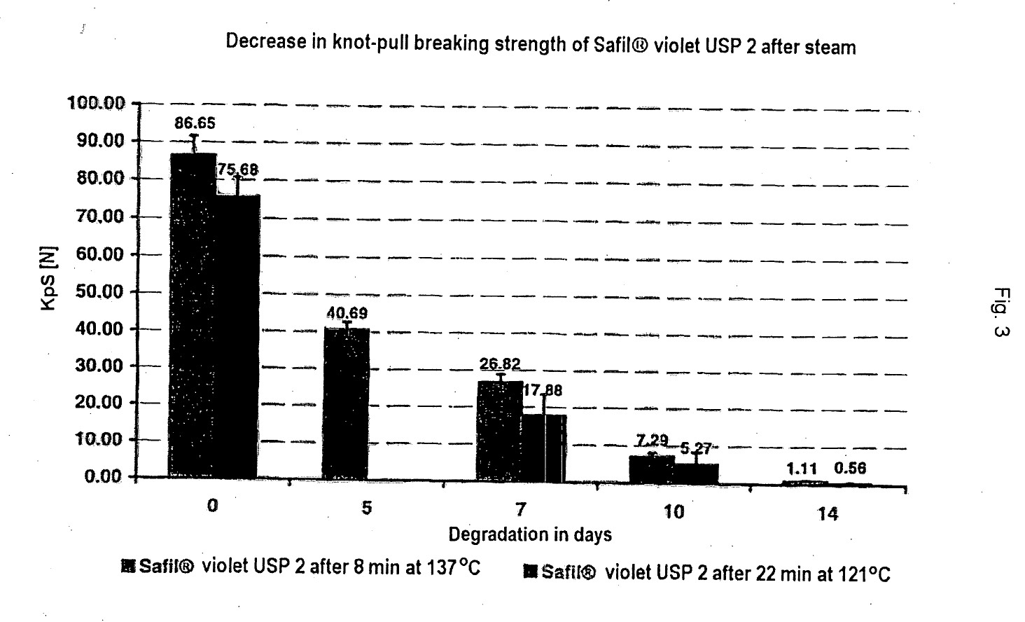

In FIG. 3 the decrease in knot-pull breaking strength of Safil® violet USP 2 after steam treatment at different temperatures.

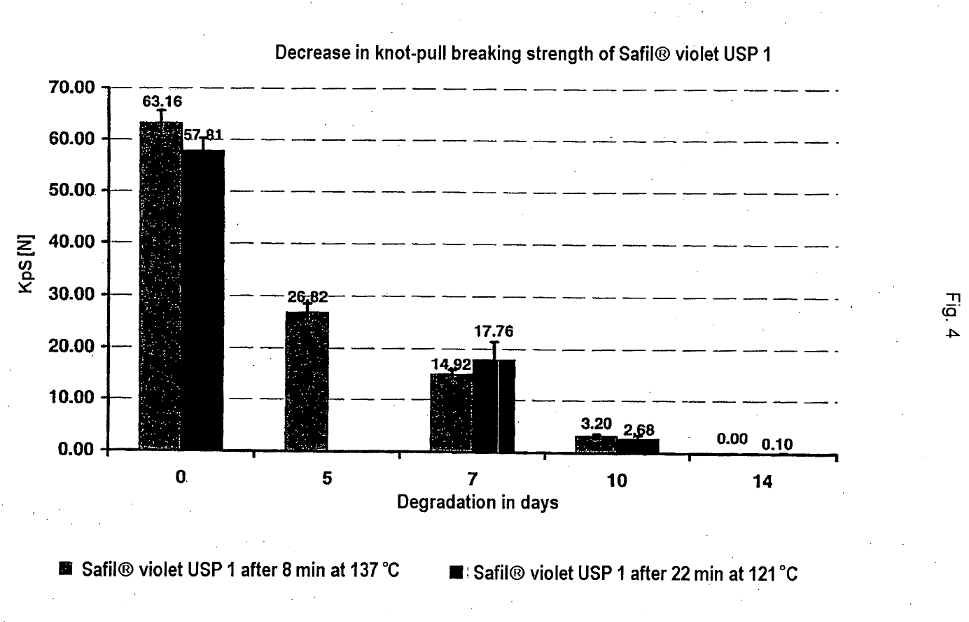

In FIG. 4 the decrease in knot-pull breaking strength of Safil® violet USP 1 after steam treatment at different temperatures.

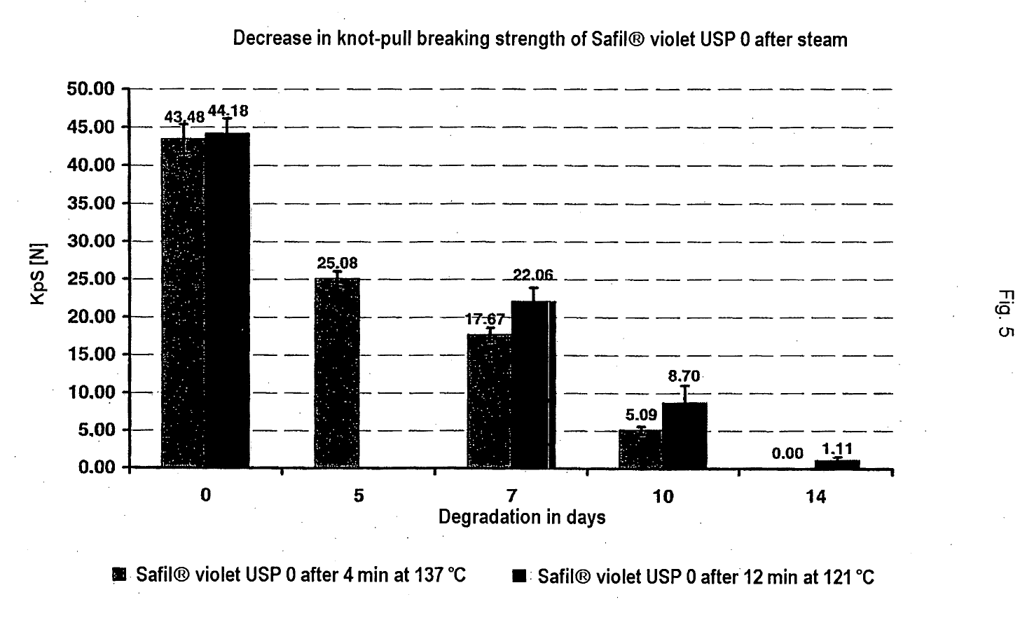

In FIG. 5 the decrease in knot-pull breaking strength of Safil® violet USP 0 after steam treatment at different temperatures.

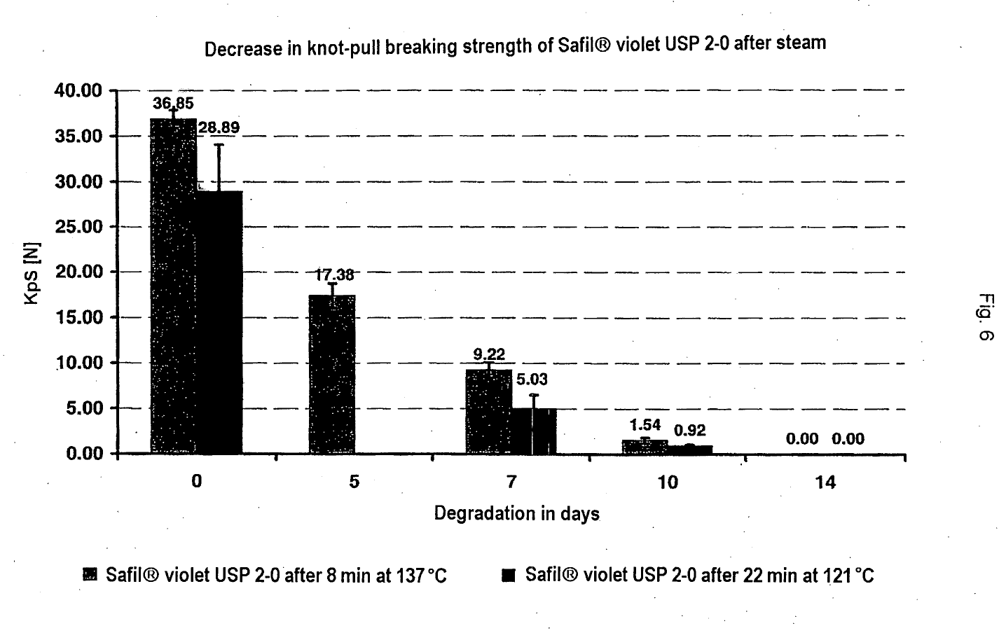

In FIG. 6 the decrease in knot-pull breaking strength of Safil® violet USP 2-0 after steam treatment at different temperatures.

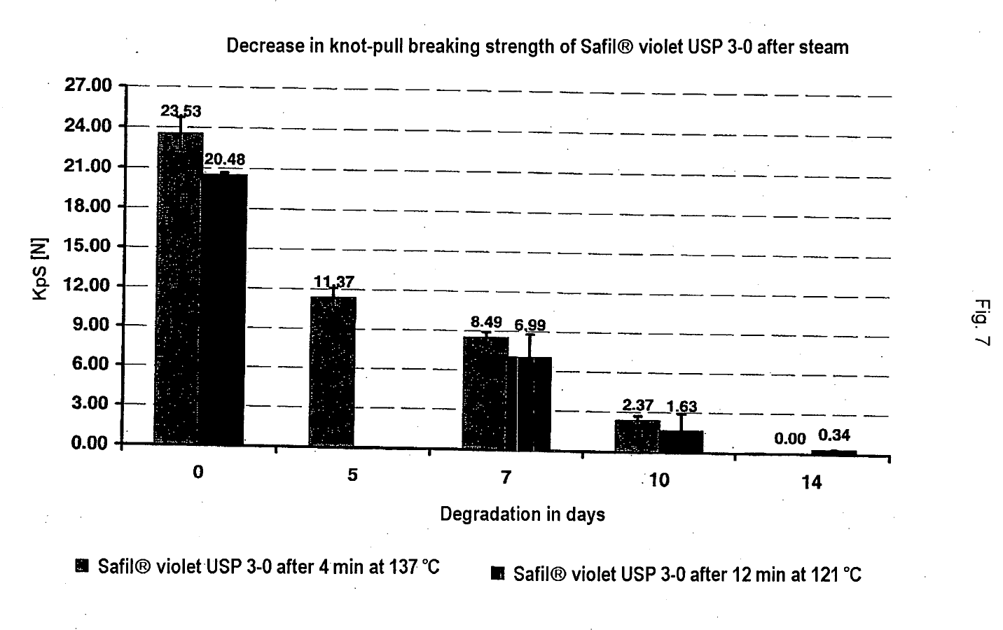

In FIG. 7 the decrease in knot-pull breaking strength of Safil® violet USP 3-0 after steam treatment at different temperatures.

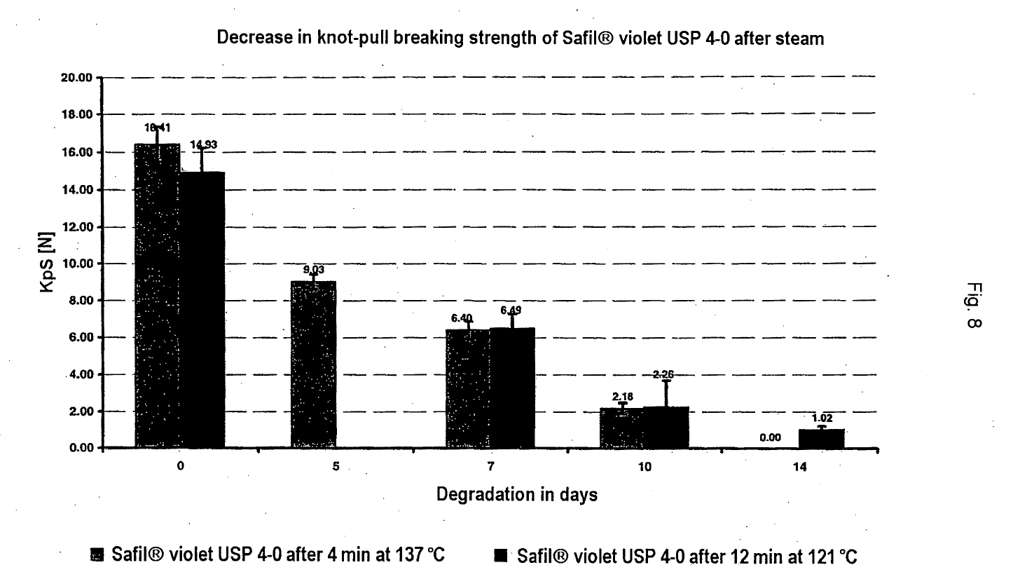

In FIG. 8 the decrease in knot-pull breaking strength of Safil® violet USP 4-0 after steam treatment at different temperatures.

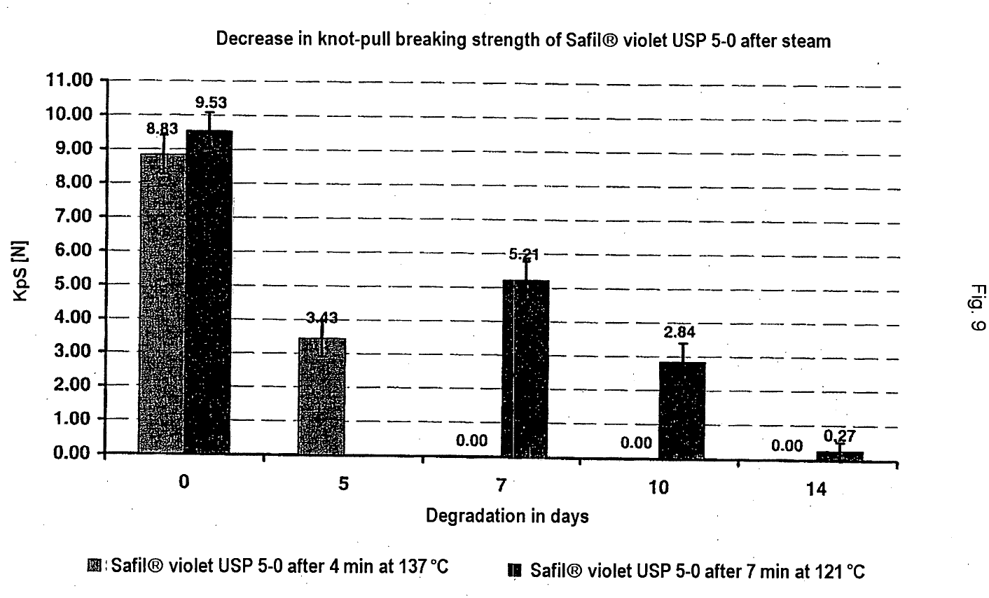

In FIG. 9 the decrease in knot-pull breaking strength of Safil® violet USP 5-0 after steam treatment at different temperatures.

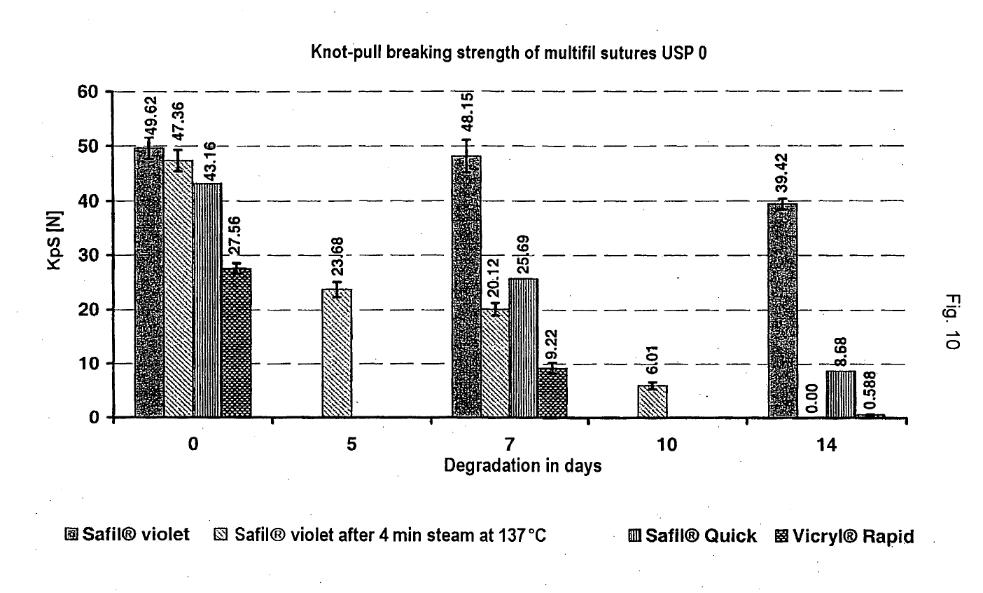

In FIG. 10 the initial knot-pull breaking strength of suture treated according to the present invention compared with untreated suture and also compared with further known absorbable sutures and also the decrease in knot-pull breaking strength for comparison.

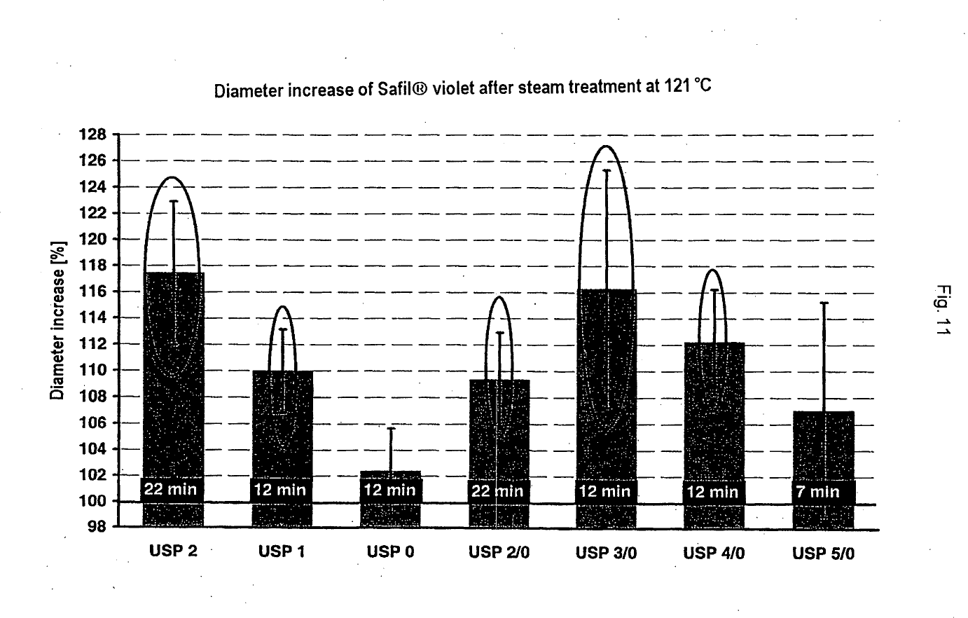

In FIG. 11 the relative diameter increase of steam-treated Safil® samples compared with untreated Safil®.

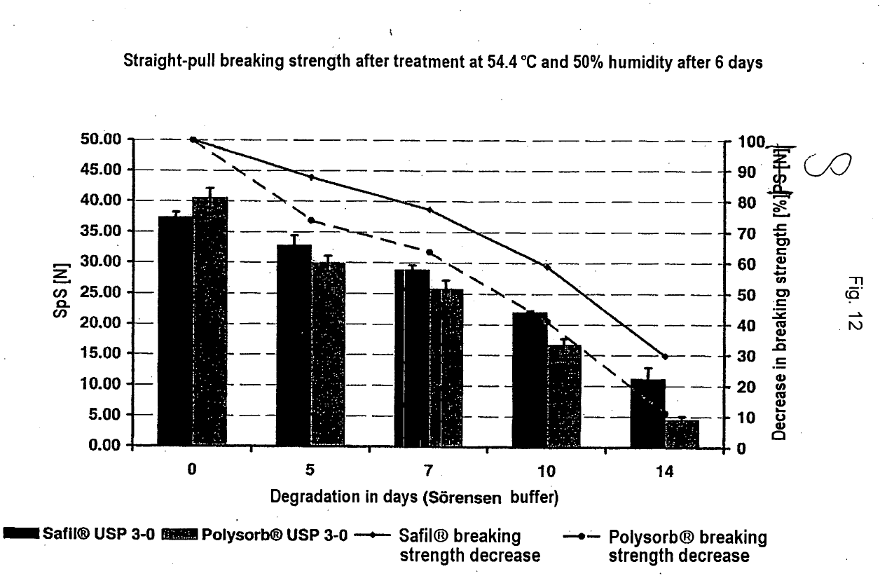

In FIG. 12 the straight-pull breaking strength of Polysorb® 3-0 and Safil® 3-0 after treatment at 54.4° C. at 50% relative humidity and the resulting degradation behavior in vivo, and the reduction in knot-pull breaking strength on immersion in a Sörensen buffering solution of pH 7.4.

### EXAMPLE

A particularly preferred embodiment of the process according to the present invention will now be described:

One or more threads to be treated are placed on a sieve and transferred into the interior of an autoclave for conducting the present invention's process. The threads comprise commercially available threads of polyglycolic acid (Safil®, for example). After introduction of the suture to be treated, the autoclave is sealed and initially evacuated. Preferably, the autoclave has already been heated at this point in time, to 121° C. in the present case. If appropriate, evacuation can be repeated, alone or combined with a flushing of the autoclave with a suitable fluid. After being evacuated once, the autoclave is filled with water vapor until it has reached its final pressure (about 2100 millibars). The pressure attained is maintained at a constant value for about 10 minutes. The temperature in the autoclave is a constant 121° C. during this period. This is followed by a second evacuation of the autoclave within about 60 seconds. The evacuation is maintained for about 8 min, during which the suture dries at unchanged temperature. Once drying is completed the treatment according to the process of the present invention stands completed. The autoclave is vented and opened and the treated suture is removed.

The steam cycle for this embodiment of the present invention's process at 121° C. is schematically depicted in FIG. 1 (pressure in mbar plotted versus time in min).

FIG. 2 illustrates the reduction in knot-pull breaking strength associated with an in vivo degradation for USP 2-0 lactide copolymer suture as described in EP 999227 A2 and treated by the process of the present invention. After having been treated according to the present invention, the suture is exposed to in vitro conditions which simulate an in vivo degradation, as already described elsewhere. Beforehand, the initial knot-pull breaking strength (after treatment but before degradation, i.e., at time 0 in FIG. 2) was determined, followed in the course of degradation by the retained knot-pull breaking strength after 14, 28, 42 and 56 days of immersion at room temperature in a Sörensen buffering solution at pH 7.4, as already described above. The upper curve in the illustration shows the degradation behavior of untreated lactide copolymer and the lower curve that of lactide copolymer treated according to the present invention. The suture of EP 999227 A2 is composed of a block copolymer consisting of 75% hard fraction and 25% soft fraction, the hard segment consisting of 95% by weight L-lactide (LLA) and 5% by weight trimethylene carbonate (TMC) and the soft segment consisting of 45% by weight ε-caprolactone (CL), 45% by weight trimethylene carbonate and 10% by weight L-lactide. The figure reveals that the knot-pull breaking strength of the steam-treated suture investigated decreases essentially linearly over a period of 56 days under in vivo conditions.

Similarly, FIGS. 3 to 9 illustrate the reduction in knot-pull breaking strength associated with a degradation in vivo for suture treated according to the present invention, albeit suture based on polyglycolic acid. FIG. 10 shows a comparison of the degradation behavior of suture treated according to the present invention with that of other sutures. The suture was in all cases exposed to the in vitro conditions already mentioned. Initial knot-pull breaking strength was determined and also the retained knot-pull breaking strength after a degradation of 5, 7, 10 and 14 days of immersion at room temperature in Sörensen buffering solution of pH 7.4. The steam treatments of the suture investigated were carried out as described above in the example. At 121° C., the steam pressure curve in all cases corresponded essentially to that described in FIG. 1. In the case of steam treatments at 137° C., the steam pressure curve measured in the autoclave shifts in accordance with the vapor pressure curve of water. Measurements indicated a final pressure of about 3.3 bar for steam treatments at 137° C.

FIG. 3 shows the decrease in the knot-pull breaking strength of Safil® violet USP 2, (the knot-pull breaking strength of which was determined to be 97.8 N before the steam treatment) after 8 min of steam treatment at 137° C. and after 22 min of steam treatment at 121° C. The dark bars on the right describe the knot-pull breaking strength of suture treated at 121° C., while the lighter bars on the left describe that of suture treated at 137° C.

FIG. 4 shows the decrease in the knot-pull breaking strength of Safil® violet USP 1, (the knot-pull breaking strength of which was determined to be 74.25 N before the steam treatment) after 8 min of steam treatment at 137° C. and after 22 min of steam treatment at 121° C. The dark bars on the right describe the knot-pull breaking strength of suture treated at 121° C., while the lighter bars on the left describe that of suture treated at 137° C.

FIG. 5 shows the decrease in the knot-pull breaking strength of Safil® violet USP 0, (the knot-pull breaking strength of which was determined to be 52.85 N before the steam treatment) after 4 min of steam treatment at 137° C. and after 12 min of steam treatment at 121° C. The dark bars on the right describe the knot-pull breaking strength of suture treated at 121° C., while the lighter bars on the left describe that of suture treated at 137° C.

FIG. 6 shows the decrease in the knot-pull breaking strength of Safil® violet USP 2-0, (the knot-pull breaking strength of which was determined to be 44.80 N before the steam treatment) after 8 min of steam treatment at 137° C. and after 22 min of steam treatment at 121° C. The dark bars on the right describe the knot-pull breaking strength of suture treated at 121° C., while the lighter bars on the left describe that of suture treated at 137° C.

FIG. 7 shows the decrease in the knot-pull breaking strength of Safil® violet USP 3-0, (the knot-pull breaking strength of which was determined to be 27.11 N before the steam treatment) after 4 min of steam treatment at 137° C. and after 12 min of steam treatment at 121° C. The dark bars on the right describe the knot-pull breaking strength of suture treated at 121° C., while the lighter bars on the left describe that of suture treated at 137° C.

FIG. 8 shows the decrease in the knot-pull breaking strength of Safil® violet USP 4-0, (the knot-pull breaking strength of which was determined to be 18.55 N before the steam treatment) after 4 min of steam treatment at 137° C. and after 12 min of steam treatment at 121° C. The dark bars on the right describe the knot-pull breaking strength of suture treated at 121° C., while the lighter bars on the left describe that of suture treated at 137° C.

FIG. 9 shows the decrease in the knot-pull breaking strength of Safil® violet USP 5-0, (the knot-pull breaking strength of which was determined to be 12 N before the steam treatment) after 4 min of steam treatment at 137° C. and after 7 min of steam treatment at 121° C. The dark bars on the right describe the knot-pull breaking strength of suture treated at 121° C., while the lighter bars on the left describe that of suture treated at 137° C. It is noticeable that in the case of the Safil® violet USP 5-0 treated according to the present invention the knot-pull breaking strength has decreased within just 7 days of degradation to values which are no longer measurable.

FIG. 10 shows the initial knot-pull breaking strength of suture treated according to the present invention (Safil® violet A60 USP 0 treated with steam at 137° C. for 4 min) compared with untreated suture (Safil® violet A60 USP 0) and also in comparison with the two sutures (both likewise USP 0) known under the trade names of Safil® Quick and Vicryl® Rapid, and also the decrease in knot-pull breaking strength for comparison. Safil® Quick comprises surgical suture of accelerated absorbability, marketed by the applicant. The knot-pull breaking strength of untreated Safil® violet is in each case depicted on the time axis as dark gray, white dotted bars (hard left at 0 days). The lighter bar immediately to the right therefrom, with inclined hatching, illustrates in each case the knot-pull breaking strength of suture treated according to the present invention. The knot-pull breaking strength of Safil® Quick is in each case depicted by the longitudinally striped bar (the third bar from the left at 0 days), while that of Vicryl® Rapid is illustrated by the bar positioned hard right at 0 days. The diagram reveals that the knot-pull breaking strength of suture treated according to the present invention decreases distinctly more rapidly than that of untreated suture. Whereas for example untreated Safil® violet still retains about 80% of its original knot-pull breaking strength after 14 days, the knot-pull breaking strength of treated Safil® violet after 14 days is no longer measurable.

FIG. 11 illustrates the increase in diameter to which suture can be subject when treated by the process of the present invention. What is shown is in each case the percentage increase in diameter increase of Safil® samples of various thread gages as it may result from a steam treatment of the samples by the process of the present invention.

FIG. 12 shows the initial straight-pull breaking strength of Polysorb® 3-0 and Safil® 3-0, in both cases after treatment in an environmental chamber in accordance with the above-cited WO 2004/050127 at 54.4° C. and 50% relative humidity. The illustration further reveals the resulting in vivo degradation behavior, or the reduction in knot-pull breaking strength on immersion in a Sörensen buffering solution at pH 7.4. The two sutures investigated exhibit similar degradation characteristics. Knot-pull breaking strength initially decreases essentially uniformly and comparatively slowly, while the decrease in knot-pull breaking strength is accelerated toward the end. This is in contradistinction to a degradation behavior as discernible in particular from FIGS. 3 to 8. There, knot-pull breaking strength, starting from a comparatively high initial value, initially decreases comparatively rapidly, only to slow down as time progresses.

We claim:

1-30. (canceled)

31. An absorbable, thermally steam-treated, surgical suture based on polyglycolic acid and/or lactide copolymer comprising rapid degradation and rapid decrease in knot-pull breaking strength under in vivo conditions and an initial knot-pull breaking strength which is high for the rapid degradation.

32. The suture based on polyglycolic acid according to claim 31, wherein the knot-pull breaking strength decreases within 5 days to less than 65% of the initial knot-pull breaking strength.

33. The suture based on polyglycolic acid according to claim 32, wherein the knot-pull breaking strength decreases within 14 days to less than 10% of the initial knot-pull breaking strength.

34. The suture based on lactide copolymer according to claim 31, wherein the knot-pull breaking strength decreases within 14 days to 75% to 90% of the initial knot pull breaking strength.

35. The suture based on lactide copolymer according to claim 34, wherein the knot-pull breaking strength decreases within 28 days to 60% of the initial knot-pull breaking strength.

36. The suture according to claim 31, wherein the initial knot-pull breaking strength is at least 65% of the knot-pull breaking strength of non-steam-treated suture.

37. The suture according to claim 31, obtainable by thermal treatment of hydrolyzable suture with water vapor.

38. The suture according to claim 37, wherein the thermal treatment is a pressurized steam treatment.

39. The suture according to claim 31, wherein the knot-pull breaking strength is between 10 N and 90 N.

40. The suture based on polyglycolic acid according to claim 31, wherein it has a viscosity number of 0.45-0.65 dl/g.

41. The suture according to claim 31, wherein it is a multifil.

42. The suture according to claim 31, wherein it has a coating.

43. A process for producing rapidly absorbable, surgical suture according to claim 31, wherein the hydrolyzable suture is subjected to a thermal treatment with water vapor.

44. The process according to claim 43, wherein the treatment is carried out with water vapor which is free of admixtures.

45. The process according to claim 43, wherein the thermal treatment is carried out at a treatment temperature in the range between 100° C. and 150° C.

46. The process according to claim 43, wherein the thermal treatment is carried out at a pressure between 1 bar and 5 bar.

47. The process according to claim 43, wherein the thermal treatment is carried out over a treatment duration of 4 min to 30 min.

48. The process according to claim 43, wherein the suture is dried after the thermal treatment.

49. The process according to claim 48, wherein the drying is carried out under reduced pressure.

50. The process according to claim 43, wherein the thermal treatment with water vapor is carried out in an autoclave.

51. The process according to claim 43, wherein the thermal treatment is carried out with water vapor essentially free of air.

52. The process according to claim 47, wherein the initial knot-pull breaking strength of the absorbable, surgical suture produced is set as a function of thread gage.

53. The process according to claim 43, wherein suture consisting essentially of polyglycolic acid is treated.

54. The process according to claim 43, wherein suture consisting essentially of lactide copolymer is treated.

55. The process according to claim 43, wherein multifil suture is treated.

56. The process according to claim 43, wherein suture treated has a coating.

57. The process according to claim 43, wherein colored suture is treated.

58. The process according to claim 57, wherein green or violet suture is treated.

59. The process according to claim 43, wherein ready-to-use suture is treated.

60. Suture obtained by a process according to claim 43.

61. Suture obtainable by a process according to claim 43.
